SEQUENCE LISTING (17 sequences)

SEQ ID NO: 1 - DNA, 18 bp, synthetic construct
  source 1..18: mol_type = other DNA
gggtagggcgggttgggt

SEQ ID NO: 2 - DNA, 25 bp, synthetic construct
  source 1..25: mol_type = other DNA
actcaacacaagaagagatcgacaa

SEQ ID NO: 3 - DNA, 21 bp, synthetic construct
  source 1..21: mol_type = other DNA
gatgagcggttgatgtccaaa

SEQ ID NO: 4 - DNA, 20 bp, synthetic construct
  source 1..20: mol_type = other DNA
ggcgtaaagcgcatgcaggt

SEQ ID NO: 5 - DNA, 22 bp, synthetic construct
  source 1..22: mol_type = other DNA
gaaattctacccccctctacag

SEQ ID NO: 6 - DNA, 1257 bp, Vibrio parahaemolyticus
  source 1..1257: mol_type = other DNA
atgatgaaaaaaacaatcacactattaactgcattactcccgcttgcttctgcagttgccgaagagccaaccttatcaccagaaatggtttcagcgtctgaagtgatcagcacgcaagaaaaccaaacctatacctatgttcgctgttggtatcgcaccagctactcgaaagatgatccggcgaccgattgggaatgggcaaaaaacgaagatggtagctacttcaccattgacggctactggtggagctccgtttcatttaaaaacatgttctacaccaacacgtcgcaaaacgttatccgtcagcgttgtgaagcaacattagatttggcgaacgagaacgcagacattacgttcttcgccgctgacaatcgcttctcatacaaccacacgatctggagcaacgacgcagcaatgcagccagatcaaatcaacaaagtggttgcactcggtgacagcttgtctgatacaggcaacatctttaacgcatcacaatggcgcttccctaacccgaacagctggttcttaggtcacttctccaacggttttgtgtggacagaatacattgccaaagcgaagaaccttccgctctacaactgggcagttggcggtgcggctggtgagaaccaatacatcgcgctaacaggggttggtgatcaagtttcttcgtacttaacctacgcaaaactggcgaagaactacaaaccagcaaacaccttgtttacgcttgagtttggtttgaatgacttcatgaactacaaccgtggcgttccagaagtgaaagcggattatgcagaagcactgattcgtttgacggacgcaggtgcgaagaacttcatgttgatgacactgccagatgcgacgaaagcgcctcagtttaagtactcaacacaagaagagatcgacaaaattcgtgcgaaagtgcttgagatgaacgagttcatcaaggcacaagcgatgtactacaaagcgcaaggttacaacatcacgttgtttgatactcacgccttgttcgagacgctaacttctgcgccagaagagcacggtttcgtgaacgcgagcgatccttgtttggacatcaaccgctcatcgtctgtcgattacatgtacacccacgcattgcgctctgagtgtgcagcgtctggtgctgagaagtttgtgttctgggatgtcacgcacccaacaacagcaactcaccgctatgttgcagagaaaatgctagaaagtagcaacaacttagaagagtttcgcttttaa

SEQ ID NO: 7 - DNA, 208 bp, Vibrio parahaemolyticus
  source 1..208: mol_type = other DNA
actcaacacaagaagagatcgacaaaattcgtgcgaaagtgcttgagatgaacgagttcatcaaggcacaagcgatgtactacaaagcgcaaggttacaacatcacgttgtttgatactcacgccttgttcgagacgctaacttctgcgccagaagagcacggtttcgtgaacgcgagcgatccttgtttggacatcaaccgctcatc

SEQ ID NO: 8 - DNA, 48 bp, synthetic construct
  source 1..48: mol_type = other DNA
gggtagggcgggttgggtaaaaaactcaacacaagaagagatcgacaa

SEQ ID NO: 9 - DNA, 44 bp, synthetic construct
  source 1..44: mol_type = other DNA
gggtagggcgggttgggtaaaaagatgagcggttgatgtccaaa

SEQ ID NO: 10 - DNA, 53 bp, synthetic construct
  source 1..53: mol_type = other DNA
aaaaagggtagggcgggttgggtaaaaaactcaacacaagaagagatcgacaa

SEQ ID NO: 11 - DNA, 49 bp, synthetic construct
  source 1..49: mol_type = other DNA
aaaaagggtagggcgggttgggtaaaaagatgagcggttgatgtccaaa

SEQ ID NO: 12 - DNA, 58 bp, synthetic construct
  source 1..58: mol_type = other DNA
aaaaaaaaaagggtagggcgggttgggtaaaaaactcaacacaagaagagatcgacaa

SEQ ID NO: 13 - DNA, 54 bp, synthetic construct
  source 1..54: mol_type = other DNA
aaaaaaaaaagggtagggcgggttgggtaaaaagatgagcggttgatgtccaaa

SEQ ID NO: 14 - DNA, 63 bp, synthetic construct
  source 1..63: mol_type = other DNA
aaaaaaaaaaaaaaagggtagggcgggttgggtaaaaaactcaacacaagaagagatcgacaa

SEQ ID NO: 15 - DNA, 58 bp, synthetic construct
  source 1..58: mol_type = other DNA
aaaaaaaaaaaaaagggtagggcgggttgggtaaaaagatgagcggttgatgtccaaa

SEQ ID NO: 16 - DNA, 63 bp, synthetic construct; identical to SEQ ID NO: 14
  source 1..63: mol_type = other DNA

SEQ ID NO: 17 - DNA, 59 bp, synthetic construct
  source 1..59: mol_type = other DNA
aaaaaaaaaaaaaaagggtagggcgggttgggtaaaaagatgagcggttgatgtccaaa

(19) United States
(12) Patent Application Publication
(10) Pub. No.: US 2026/0286450 A1
(43) Pub. Date: Sep. 24, 2026

(54) DETECTION METHOD USING HRPZYME

(71) Applicants: Ali Parsaeimehr, Dover, DE (US); Gulnihal Ozbay, Clayton, DE (US)

(72) Inventors: Ali Parsaeimehr, Dover, DE (US); Gulnihal Ozbay, Clayton, DE (US)

(73) Assignee: Delaware State University, Dover, DE (US)

(21) Appl. No.: 19/476,585

(22) PCT Filed: Apr. 19, 2024
(86) PCT No.: PCT/US2024/025338

§ 371 Date: Oct. 17, 2025

Related U.S. Application Data
(60) Provisional application No. 63/460,756, filed on Apr. 20, 2023.

(51) Int. Cl. C12Q 1/689; C12Q 1/6809; C12Q 1/686; G01N 21/29

(52) U.S. Cl. CPC C12Q 1/689; C12Q 1/6809; C12Q 1/686; G01N 21/293; C12Q 2600/166

(57) ABSTRACT

The present invention provides a colorimetric detection platform for detecting a target polynucleotide using specific primers for the target polynucleotide coupled with the HRPzyme sequence of GGGTAGGGCGGGTTGGGT (SEQ ID NO: 1). Also provided are related detection methods, kits and compositions.

### CROSS-REFERENCE TO RELATED APPLICATION

This application claims priority to U.S. Provisional Application No. 63/460,756, filed Apr. 20, 2023, and the contents of which are incorporated herein by reference in their entireties for all purposes.

### REFERENCE TO U.S. GOVERNMENT SUPPORT

This invention was made with government support under grant number 1757353 awarded by the Established Program to Stimulate Competitive Research (EPSCoR) of the U.S. National Science Foundation (NSF). The United States has certain rights in the invention.

### REFERENCE TO SEQUENCE LISTING

The present application is being filed along with a Sequence Listing in electronic format. The Sequence Listing is provided as a file entitled Sequence Listing_DSU1-119WO.xml, created Apr. 19, 2024, which is 17,000 bites in size. The information in XML file format of the Sequence Listing is incorporated herein by reference in its entirety.

### FIELD OF THE INVENTION

The invention relates to the use of HRPzyme-integrated primers for detecting a target DNA polynucleotide in a sample.

### BACKGROUND OF THE INVENTION

The world today is increasingly connected in terms of food security, safety, and the outbreak of foodborne illnesses. This issue has been intensified by globalization and the expansion of international food trade. According to the World Health Organization (WHO), an estimated 600 million cases of foodborne illness and 420,000 fatalities occur annually due to the consumption of contaminated food products. Among foodborne pathogens, Vibrio parahaemolyticus, a Gram-negative bacterium, significantly contributes to worldwide foodborne morbidity and mortality. This halophilic bacterium is commonly found in marine and estuarine environments across the globe and is frequently isolated from various seafood products. Evidence indicates that V. parahaemolyticus colonizes the human gut and is responsible for acute gastroenteritis when raw or undercooked seafood is consumed. Ensuring the safety of our sea food supplies critically depends on the rapid and accurate detection of V. parahaemolyticus and while traditional culture-based methods are still in use for detecting Vibrio species, they often struggle to accurately identify the isolated species. Consequently, molecular techniques, such as 16S rRNA, ELISA, isotyping, and antisera are gaining popularity to overcome the limitations of culture-based approaches in pathogen detection. In this context, virulence genes (Vir genes) have emerged as crucial molecular markers for pathogen detection. In V. parahaemolyticus, numerous Vir genes, including tlh (thermolabile hemolysin), tdh (thermostable direct hemolysin), and trh (tdh-related hemolysin), have been identified and sequenced from isolated V. parahaemolyticus strains. These genes have been targeted for molecular detection using various clinical and laboratory assays, such as polymerase chain reaction (PCR), reverse transcription-polymerase chain reaction (RT-PCR), and enzyme-linked immunosorbent assay (ELISA). Despite their accuracy and efficiency, these techniques are often characterized by high costs, lengthy procedures, complexity, and the use of toxic fluorescence dyes like ethidium bromide. Therefore, there is an urging need for innovative, cost-effective, and eco-friendly approaches to enable large on-site screenings, particularly in resource-poor rural areas.

Colorimetric analysis of PCR products has been introduced. This method involves the addition of horseradish peroxidase-mimicking DNAzymes (HRPzymes) at the 5′ end of the primers. HRPzymes facilitate sequence-specific hybridization, forming G-quadruplex nucleotide sequences that mimic peroxidase activity upon binding to hemin. This mimicry allows for visual detection when the DNAzymes interact with molecules like luminol, 2,2′-azino-bis(3-ethylbenzothiazoline-6-sulfonic acid) (ABTS), and 3,3′,5,5′-tetramethylbenzidine (TMB) in the presence of hydrogen peroxide (H₂O₂). The short HRPzyme sequence (GGGTAGGGCGGGTTGGGT) (SEQ ID NO: 1) is designed to allow primer integration in such a way that the functional HRPzyme sequence forms only after the successful amplification of the target genomic region (Ahmad et al., 2021, Biosensors and Bioelectronics, 187, Article 113280). The HRPzyme-based detection method has demonstrated both specificity and sensitivity, enabling the detection of pathogens like Noroviruses (NoVs) at low concentrations (10 copies per mL). It is effective in detecting various other pathogens, including Escherichia coli, Vibrio parahaemolyticus, Listeria monocytogenes, Salmonella enterica, and Bacillus cereus.

Therefore, there remains a need for an on-site sensitive and rapid method for detecting pathogens (e.g., bacteria).

### SUMMARY OF THE INVENTION

The present invention relates to detection of a target polynucleotide in a sample using primers containing the functional HRPzyme sequence of GGGTAGGGCGGGTTGGGT (SEQ ID NO: 1). The invention is based on the inventors' discovery of a colorimetric detection platform using specific primers targeting the thermolabile hemolysin (tlh) region in V. parahaemolyticus, coupled with the HRPzyme sequence. This innovative technique is characterized by its sensitivity and rapidity, eliminating the need for gel electrophoresis. It can be conveniently employed for on-site detection of V. parahaemolyticus in seafood products, such as Eastern Oyster (Crassostrea virginica) and Blue Crab (Callinectes sapidus).

The present invention provides a method for detecting a target DNA polynucleotide in a sample. The method comprises: (a) obtaining a target DNA polynucleotide from a sample and a control DNA polynucleotide, wherein the target DNA polynucleotide comprises a target DNA sequence, and wherein the control DNA polynucleotide lacks the target DNA sequence; (b) providing a first primer and a second primer, wherein the first primer comprises a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer, wherein the second primer comprises a second protector, the HRPzyme sequence, a second spacer and a reverse primer sequence for the target DNA sequence in the order from the 5′ end to the 3′ end of the second primer; (c) performing a polymerase chain reaction (PCR) in a test mixture, wherein the test mixture comprises the target DNA polynucleotide, the first primer, and the second primer, whereby a PRC product is produced in the test mixture; (d) exposing the test mixture from step (c) to hemin, a substrate and hydrogen peroxide; (e) making a test color measurement in the test mixture from step (d); (f) performing a PCR in a control mixture, wherein the control mixture comprises the control DNA polynucleotide, the first primer, and the second primer, whereby no PRC product is produced in the control mixture; (g) exposing the control mixture from step (f) to hemin, a substrate and hydrogen peroxide; (h) making a control color measurement in the control mixture from step (g); and (i) comparing the test color measurement with the control color measurement, wherein a color difference between the test color measurement and the control color measurement indicates the presence of the target DNA polynucleotide in the sample. The target DNA sequence may be a gene or a fragment thereof specific to a bacterium. The gene may be the tlh gene specific to Vibrio parahaemolyticus. The forward primer sequence may be ACTCAACACAAGAAGAGATCGACAA (SEQ ID NO: 2) and the reverse primer sequence may be GATGAGCGGTTGATGTCCAAA (SEQ ID NO: 3).

Each of the first protector and the second protector may be independently a polyadenine having 1-50 nucleotides. The first protector and the second protector may be the same.

Each of the first spacer and the second spacer may be independently a polyadenine having a length of 1-20 nucleotides. In one embodiment, the first spacer is a polyadenine having 5-7 nucleotides and the second spacer is a polyadenine having 4-6 nucleotides.

The test mixture may comprise the first primer and the second primer at a combined concentration of 1-1000 nM before the PCR reaction is performed in the test mixture.

The substrate may be selected from the group consisting of luminol, 2,2′-azino-bis(3-ethylbenzothiazoline-6-sulfonic acid) (ABTS) and 3,3′,5,5′-tetramethylbenzidine (TMB). The substrate may be 2,2′-azino-bis(3-ethylbenzothiazoline-6-sulfonic acid) (ABTS).

The test sample may comprise Vibrio parahaemolyticus at a concentration of 1-100 colony-forming units (CFU) per mL.

The sample may be obtained from an organism. The organism may be Crassostrea virginica. The organism may be Callinectes sapidus.

The method may further comprise detecting the color change by a naked eye.

The method may further comprise detecting the color change within 0.5-3 hours after the target DNA polynucleotide is obtained from the sample.

For each method of the present invention, a kit is provided for detecting a target DNA polynucleotide in a sample. The target DNA polynucleotide comprises a target DNA sequence. The kit comprises (a) a first primer comprising a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer, (b) a second primer comprising a second protector, the HRPzyme sequence, a second spacer and a reverse primer sequence for the target DNA sequence in the order from the 5′ end to the 3′ end of the second primer, and (c) an instruction for performing the method. The kit may further comprise one or more agents selected from the group consisting of a control DNA polynucleotide, hemin, a substrate, and hydrogen peroxide.

The present invention further provides a composition for detecting a target DNA sequence. The composition comprises a first primer and a second primer, wherein the first primer comprises a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer, wherein the second primer comprises a second protector, the HRPzyme sequence, a second spacer and a reverse primer sequence for the target DNA sequence in the order from the 5′ end to the 3′ end of the second primer. The target DNA sequence may encode the tlh gene or a fragment thereof specific to Vibrio parahaemolyticus, wherein the forward primer sequence is ACTCAACACAAGAAGAGATCGACAA (SEQ ID NO: 2) and the reverse primer sequence is GATGAGCGGTTGATGTCCAAA (SEQ ID NO: 3). Each of the first protector and the second protector may be independently a polyadenine having 1-50 nucleotides. The first protector and the second protector may be the same.

Each of the first spacer and the second spacer may be independently a polyadenine having a length of 1-20 nucleotides. The first spacer may be a polyadenine having 6 nucleotides and the second spacer may be a polyadenine having 5 nucleotides. The composition may comprise the first primer and the second primer at a combined concentration of 1-1000 nM.

### BRIEF DESCRIPTION OF THE DRAWINGS

FIGS. 1a-c show molecular confirmation of Vibrio parahaemolyticus using specific primers. (a) molecular confirmation of V. parahaemolyticus using 16s rRNA, (b) molecular confirmation of V. parahaemolyticus using specific primer for detection of thermolabile hemolysin (tlh) gene. (c) the confirmation of V. parahaemolyticus using RT-PCR. The Thiosulfate-Citrate-Bile Salts-Sucrose (TCBS) Agar was used for growth of V. parahaemolyticus. The sequence of 16s rRNA primer, F: GGCGTAAAGCGCATGCAGGT (SEQ ID NO: 4), R: GAAATTCTACCCCCCTCTACAG (SEQ ID NO: 5). The sequence of tlh primers: F: ACTCAACACAAGAAGAGATCGACAA (SEQ ID NO: 2); R: GATGAGCGGTTGATGTCCAAA (SEQ ID NO: 3). PCR condition: 16S rRNA: denaturation at 95° C. for 3 min, followed by 34 cycles of denaturation at 95° C. for 30 sec, annealing at 58° C. for 30 sec, and primer extension at 72° C. for 1 min; followed by final extension at 72° C. for 5 min. tlh: denaturation at 95° C. for 3 min, followed by 35 cycles of denaturation at 95° C. for 30 sec, annealing at 60° C. for 40 sec, and primer extension at 72° C. for 1 min; followed by final extension at 72° C. for 10 min.

FIGS. 2a-d show optimization of the primers used for the sensitive detection of Vibrio parahaemolyticus using the colorimetric HRPzyme-integrated PCR assay. (a-b) the influence of primer concentration (nM), (c-d) the influence of protector's length (nt). 10 nt protector length sequence (Relative intensity=3.07±0.23) was determined as the length for assay. 500 nM was also determined as the best primer concentration (Relative intensity=3.14±0.31). PCR condition: denaturation at 95° C. for 3 min, incubation time at 60° C. for 30 min, followed by 30 cycles of denaturation at 95° C. for 15 sec, annealing at 60° C. for 10 sec, and primer extension at 72° C. for 10 sec; followed by final extension at 72° C. for 5 min. Errors bars are representing the standard deviation among 6 different experiments (replicates=3).

FIGS. 3a-d show influence of annealing temperature and incubation time on the Colorimetric HRPzyme-integrated PCR assay for detection of Vibrio parahaemolyticus (a-b) a temperature dependence activity was observed on the colorimetric signal starting at Ta=50° C. (PCR condition: denaturation at 95° C. for 3 min, incubation time at 60° C. for 30 min, followed by 30 cycles of denaturation at 95° C. for 15 sec, annealing at 60° C. for 10 sec, and primer extension at 72° C. for 10 sec; followed by final extension at 72° C. for 5 min) (c-d) alternation in colorimetric signals was observed (in each set of bars, left bar: -; right bar: +) at different denaturation stage using different incubation times (5, 10, 15, 30, 45, 60 min). No difference in colorimetric signal was observed after 30 min incubation time at 60° C. Errors bars are representing the standard deviation among 6 different experiments (replicates=3).

FIGS. 4a-c show colorimetric HRPzyme-integrated PCR assay results of Vibrio parahaemolyticus. (a) gel electrophoresis data of different concentration of V. parahaemolyticus, (b) colorimetric HRPzyme-integrated PCR results of different concentration of V. parahaemolyticus, (c) absorbance data of colorimetric HRPzyme-integrated PCR assay at different concentration of bacteria. PCR condition: denaturation at 95° C. for 3 min, incubation time at 60° C. for 30 min, followed by 30 cycles of denaturation at 95° C. for 15 sec, annealing at 60° C. for 10 sec, and primer extension at 72° C. for 10 sec; followed by final extension at 72° C. for 5 min. Errors bars are representing the standard deviation among 6 different experiments (replicates=3).

FIGS. 5a-c show a specificity test of Colorimetric HRPzyme-integrated PCR assay results of Vibrio parahaemolyticus. (a) gel electrophoresis data on the specificity test of V. parahaemolyticus against tested pathogens, (b) colorimetric HRPzyme-integrated PCR results (c) absorbance data of colorimetric HRPzyme-integrated PCR assay (OD=410 nm). Errors bars are representing the standard deviation among 6 different experiments. Colorimetric PCR-based detection method. The samples were run at 60° C. for a duration of 60 min and read at the OD=410 nm. 100 bp ladder was used for molecular confirmation. Data is presenting based on 10 independent experiments, replicates=3.

FIGS. 6a-f show colorimetric HRPzyme-integrated PCR assay results of Vibrio parahaemolyticus on Oyster. (a) Oyster, (b, f) colorimetric HRPzyme-integrated PCR results of different concentration of V. parahaemolyticus, (c, e) molecular confirmation on the presence of the V. parahaemolyticus using gel electrophoresis data of different concentration of V. parahaemolyticus (d) the RT-PCR confirmation on the presence of the V. parahaemolyticus. Note: (1) adductor muscle, (2) heart, (3) pericardial cavity, (4) gonad area, and (5) the gills parts. Data is presenting based on 10 independent experiments, replicates=3.

FIGS. 7a-d show colorimetric HRPzyme-integrated PCR assay results of Vibrio parahaemolyticus on Blue Crab. (a) isolating hemolymph from the Blue crab, (b, f) colorimetric HRPzyme-integrated PCR results of different concentration of V. parahaemolyticus, (c, e) molecular confirmation on the presence of the V. parahaemolyticus using gel electrophoresis data of different concentration of V. parahaemolyticus (d) the RT-PCR confirmation on the presence of the V. parahaemolyticus. Data is presenting based on 10 independent experiments, replicates=3.

FIG. 8 shows the DNA sequence of the tlh region in Vibrio parahaemolyticus.

The underlined sequence ACTCAACACAAGAAGAGATCGACAAAATTCGTGCGAAAGTGCTTGAGATGAACGAGTTCATCAA GGCACAAGCGATGTACTACAAAGCGCAAGGTTACAACATCACGTTGTTTGATACTCACGCCTTG TTCGAGACGCTAACTTCTGCGCCAGAAGAGCACGGTTTCGTGAACGCGAGCGATCCTTGTTTG GACATCAACCGCTCATC (SEQ ID NO: 7) is the targeted region, and the forward primer sequence is ACTCAACACAAGAAGAGATCGACAA (SEQ ID NO: 2) and the reverse primer sequence is GATGAGCGGTTGATGTCCAAA (SEQ ID NO: 3).

FIGS. 9a-b show the schematic demonstration of the HRPzyme-based colorimetric PCR method for Vibrio parahaemolyticus detection using thermolabile hemolysin (tlh) region. (a) the steps on the colorimetric detection scheme (b) the strategy used to design the colorimetric HRPzyme-integrated PCR sequence. V. parahaemolyticus was cultured using a TCBA plate and the obtained bacterial colonies was confirmed using 16SrRNA, and the tlh. The confirmed colonies were cultured in LB liquid media and used for the optimization of the test. The positive samples resulted in a signal off after ABTS test (A) and negative samples resulted in a signal on (B).

### DETAILED DESCRIPTION OF THE INVENTION

The present invention relates to a sensitive, rapid, and cost-effective colorimetric detection method involving the use of HRPzyme-integrated primers to amplify both a target DNA polynucleotide and the HRPzyme sequence. The method of the present invention is useful for on-site detection of pathogens (e.g., bacteria) in biological samples obtained from organisms, for example, in food products. The inventors have developed a PCR-based detection platform integrated with HRPzyme and utilizing DNAzyme as a signaling probe which mimics peroxidase activity, and has surprisingly discovered that the colorimetric signal is detectable at concentrations as low as 101 cfu mL-1 when measured with a spectrophotometer and at 10³ cfu ml⁻¹ through visual inspection. The inventors have also discovered that extending the polyadenine length to 10 nucleotides resulted in a significant reduction in the background signaling of HRPzyme activity. The inventors have further discovered that, even after a 120-minute incubation period, there were no further changes observed in the colorimetric signal in positive samples.

The present invention provides a method for detecting a target DNA polynucleotide in a sample. The method comprises obtaining a target DNA polynucleotide from a sample and a control DNA polynucleotide. The target DNA polynucleotide comprises a target DNA sequence while the control DNA polynucleotide lacks the target DNA sequence. The target DNA polynucleotide and the control DNA polynucleotide may have the same nucleotide sequence except the target DNA sequence. The method also comprises providing a first primer and a second primer. The first primer comprises a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer. The second primer comprises a second protector, the HRPzyme sequence, a second spacer and a reverse primer sequence for the target DNA sequence in the order from the 5′ end to the 3′ end of the second primer. The method further comprises performing a polymerase chain reaction (PCR) in a test mixture, which test mixture comprises the target DNA polynucleotide, the first primer, and the second primer such that a PRC product is produced in the test mixture; exposing the test mixture to hemin, a substrate and hydrogen peroxide after the PCR; and making a test color measurement in the test mixture after the exposure. The method further comprises performing a PCR in a control mixture, which control mixture comprises the control DNA polynucleotide, the first primer, and the second primer such that a PRC product is not produced in the control mixture; exposing the control mixture to hemin, a substrate and hydrogen peroxide after the PCR; and making a control color measurement in the control mixture after the exposure. The HRPzyme sequence may be amplified in the test mixture. The HRPzyme sequence may not be amplified in the control mixture. The method further comprises comparing the test color measurement with the control color measurement. A color difference between the test color measurement and the control color measurement indicates the presence of the target DNA polynucleotide in the sample.

The color measurement may be made qualitatively or quantitatively. The color measurement may be made using any conventionally known methods. The first color measurement and the second color measurement may be made using the same method.

The color difference between the test color measurement and the control color measurement may be visible to a naked eye. For example, a green-blue colored product may be observed in the control mixture with hemin and hydrogen peroxide while a dark to light green-blue color may be observed in the test mixture with hemin and hydrogen peroxide. The method may further comprise detecting the color change by a naked eye.

The target DNA sequence may be a gene or a fragment thereof specific to a pathogen. The presence of such a target DNA may indicate the presence of the pathogen. The pathogen may be a bacterium, for example, Vibrio parahaemolyticus, E. coli, S. aureus, or S. enterica. The gene may be the tlh gene specific to Vibrio parahaemolyticus, the 16s rRNA gene specific to E. coli, the nuc A gene specific to S. aureus, or the inv A gene specific to S. enterica. In one embodiment, the target DNA sequence is the tlh gene or a fragment thereof specific to Vibrio parahaemolyticus, and presence of the tlh gene or a fragment thereof in a sample indicates the presence of the Vibrio parahaemolyticus in the sample.

The forward primer sequence may consist of a DNA sequence at the 5′ end of the target DNA sequence while the reverse primer sequence may consist of a DNA sequence complementary to a DNA sequence at the 3′ end of the target DNA sequence. The forward primer sequence may have 12-30 nucleotides. The reverse primer sequence may have 12-30 nucleotides. Where the target DNA sequence is the tlh gene or a fragment thereof, the forward primer sequence may be ACTCAACACAAGAAGAGATCGACAA (SEQ ID NO: 2) and the reverse primer sequence may be GATGAGCGGTTGATGTCCAAA (SEQ ID NO: 3) (FIG. 8).

The first protector may be a polyadenine having about 1-5, 1-10, 1-15, 1-20, 1-25, 1-30, 1-35, 1-40, 1-45, 1-50, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45 or 20-50 nucleotides. The second protector may be a polyadenine having about 1-5, 1-10, 1-15, 1-20, 1-25, 1-30, 1-35, 1-40, 1-45, 1-50, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45 or 20-50 nucleotides. The first protector and the second protector may be the same. The first protector and the second protector may be different.

The first spacer may be a polyadenine having a length of about 1-2, 1-3, 1-4, 1-5, 1-6, 1-7, 1-8, 1-9, 1-10, 1-15, 1-20, 2-3, 2-4, 2-5, 2-6, 2-7, 2-8, 2-9, 2-10, 2-15, 2-20, 3-4, 3-5, 3-6, 3-7, 3-8, 3-9, 3-10, 3-15, 1-20, 4-5, 4-6, 4-7, 4-8, 4-9, 4-10, 4-15, 4-20, 5-6, 5-7, 5-8, 5-9, 5-10, 5-15, 5-20, 6-7, 6-8, 6-9, 6-10, 6-20, 7-8, 7-9, 7-10, 7-15, 7-20, 8-9, 8-10, 8-15, 8-20, 9-10, 9-15, 9-20, 10-15, 10-20 or 15-20 nucleotides. The second spacer may be a polyadenine having a length of about 1-2, 1-3, 1-4, 1-5, 1-6, 1-7, 1-8, 1-9, 1-10, 1-15, 1-20, 2-3, 2-4, 2-5, 2-6, 2-7, 2-8, 2-9, 2-10, 2-15, 2-20, 3-4, 3-5, 3-6, 3-7, 3-8, 3-9, 3-10, 3-15, 1-20, 4-5, 4-6, 4-7, 4-8, 4-9, 4-10, 4-15, 4-20, 5-6, 5-7, 5-8, 5-9, 5-10, 5-15, 5-20, 6-7, 6-8, 6-9, 6-10, 6-20, 7-8, 7-9, 7-10, 7-15, 7-20, 8-9, 8-10, 8-15, 8-20, 9-10, 9-15, 9-20, 10-15, 10-20 or 15-20 nucleotides. The first spacer and the second spacer may be the same. The first spacer and the second spacer may be different. In one embodiment, the first spacer is a polyadenine having 5-7 nucleotides and the second spacer is a polyadenine having 4-6 nucleotides.

The test mixture may comprise the first primer and the second primer at a combined concentration of about 0.1-100, 0.1-500, 0.1-750, 0.1-1,000, 0.1-5,000, 0.1-10,000, 1-100, 1-500, 1-750, 1-1,000, 1-5,000, 1-10,000, 5-100, 5-500, 5-750, 5-1,000, 5-5,000, 5-10,000, 10-100, 10-500, 10-750, 10-1,000, 10-5,000, 10-10,000, 15-100, 15-500, 15-750, 15-1,000, 15-5,000, 15-10,000, 20-100, 20-500, 20-750, 20-1,000, 20-5,000, 20-10,000, 100-500, 100-750, 100-1,000, 100-5,000, 100-10,000, 250-500, 250-750, 250-1,000, 250-5,000, 250-10,000, 500-750, 500-1,000, 500-5,000, 500-10,000, 1,00-5,000 or 1,000-10,000 nM before the PCR reaction is performed in the test mixture. The control mixture may comprise the first primer and the second primer at a combined concentration of about 0.1-100, 0.1-500, 0.1-750, 0.1-1,000, 0.1-5,000, 0.1-10,000, 1-100, 1-500, 1-750, 1-1,000, 1-5,000, 1-10,000, 5-100, 5-500, 5-750, 5-1,000, 5-5,000, 5-10,000, 10-100, 10-500, 10-750, 10-1,000, 10-5,000, 10-10,000, 15-100, 15-500, 15-750, 15-1,000, 15-5,000, 15-10,000, 20-100, 20-500, 20-750, 20-1,000, 20-5,000, 20-10,000, 100-500, 100-750, 100-1,000, 100-5,000, 100-10,000, 250-500, 250-750, 250-1,000, 250-5,000, 250-10,000, 500-750, 500-1,000, 500-5,000, 500-10,000, 1,00-5,000 or 1,000-10,000 nM before the PCR reaction is performed in the control mixture. The combined concentration of the first primer and the second primer in the test mixture and that in the control mixture may be the same or substantially identical, for example, at least 90%, 95% or 99% identical.

The substrate may be selected from the group consisting of luminol, 2,2′-azino-bis(3-ethylbenzothiazoline-6-sulfonic acid) (ABTS), 3,3′,5,5′-tetramethylbenzidine (TMB) and o-phenylenediamine (OPD). For example, the substrate may be 2,2′-azino-bis(3-ethylbenzothiazoline-6-sulfonic acid) (ABTS).

The test sample may comprise a bacterium at a concentration of about 1-10, 1-50, 1-100, 10-50, 10-100 or 50-100 colony-forming units (CFU) per mL. The target DNA sequence may be specific to the bacterium. The bacterium may be Vibrio parahaemolyticus. The test sample may comprise Vibrio parahaemolyticus at a concentration of about 1-10, 1-50, 1-100, 10-50, 10-100 or 50-100 colony-forming units (CFU) per mL.

The sample may be obtained from an organism. The organism may be a Crassostrea virginica or Callinectes sapidus. The organism may be in a food product, for example, seafood product. The sample may be subject to the method of the present invention after being obtained from an organism without storage at, for example, a temperature of about 0-40, 4-40, 0-37, 4-37, 0-25, 4-25, 0-20, 4-20 or 0-4° C., for a time period of at least about 1, 2, 3, 4, 5, 6, 7, 14, 21 or 28 day, or about 1-7, 1-14, 1-21, 1-28, 1-30 or 1-60 days.

The method may further comprise detecting the color change within about 0.1-0.5, 0.1-1, 0.1-2, 0.1-3, 0.1-6, 0.1-12, 0.1-18, 0.1-24, 0.5-1, 0.5-2, 0.5-3, 0.5-6, 0.5-12, 0.5-18, 0.5-24, 1-2, 1-3, 1-6, 1-12, 1-18 or 1-24 hours after the target DNA polynucleotide is obtained from the sample.

For each method of the present invention, a kit for detecting a target DNA polynucleotide in a sample is provided. The target DNA polynucleotide comprises a target DNA sequence. The kit comprises a first primer, a second primer, and an instruction for performing a method of the present invention. The first primer comprises a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer. The second primer comprises a second protector, the HRPzyme sequence, a second spacer and a reverse primer sequence for the target DNA sequence in the order from the 5′ end to the 3′ end of the second primer. The kit may further comprise one or more agents selected from the group consisting of a control DNA polynucleotide, hemin, a substrate, and hydrogen peroxide. For example, the kit may comprise a first primer, a second primer, an instruction for performing a method of the present invention, hemin, a substrate, and hydrogen peroxide.

According to the kit of the present invention, the target DNA sequence may be a gene or a fragment thereof specific to a pathogen. The pathogen may be a bacterium, for example, Vibrio parahaemolyticus, E. coli, S. aureus, or S. enterica. The gene may be the tlh gene specific to Vibrio parahaemolyticus, the 16s rRNA gene specific to E. coli, the nuc A gene specific to S. aureus, or the inv A gene specific to S. enterica. In one embodiment, the target DNA sequence is the tlh gene or a fragment thereof specific to Vibrio parahaemolyticus, and presence of the tlh gene or a fragment thereof in a sample indicates the presence of the Vibrio parahaemolyticus in the sample.

In the kit, the forward primer sequence may consist of a DNA sequence at the 5′ end of the target DNA sequence while the reverse primer sequence may consist of a DNA sequence complementary to a DNA sequence at the 3′ end of the target DNA sequence. The forward primer sequence may have 12-30 nucleotides. The reverse primer sequence may have 12-30 nucleotides. Where the target DNA sequence is the tlh gene or a fragment thereof, the forward primer sequence may be ACTCAACACAAGAAGAGATCGACAA (SEQ ID NO: 2) and the reverse primer sequence may be GATGAGCGGTTGATGTCCAAA (SEQ ID NO: 3) (FIG. 8).

In the kit, the first protector may be a polyadenine having about 1-5, 1-10, 1-15, 1-20, 1-25, 1-30, 1-35, 1-40, 1-45, 1-50, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45 or 20-50 nucleotides. The second protector may be a polyadenine having about 1-5, 1-10, 1-15, 1-20, 1-25, 1-30, 1-35, 1-40, 1-45, 1-50, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45 or 20-50 nucleotides. The first protector and the second protector may be the same. The first protector and the second protector may be different.

In the kit, the first spacer may be a polyadenine having a length of about 1-2, 1-3, 1-4, 1-5, 1-6, 1-7, 1-8, 1-9, 1-10, 1-15, 1-20, 2-3, 2-4, 2-5, 2-6, 2-7, 2-8, 2-9, 2-10, 2-15, 2-20, 3-4, 3-5, 3-6, 3-7, 3-8, 3-9, 3-10, 3-15, 1-20, 4-5, 4-6, 4-7, 4-8, 4-9, 4-10, 4-15, 4-20, 5-6, 5-7, 5-8, 5-9, 5-10, 5-15, 5-20, 6-7, 6-8, 6-9, 6-10, 6-20, 7-8, 7-9, 7-10, 7-15, 7-20, 8-9, 8-10, 8-15, 8-20, 9-10, 9-15, 9-20, 10-15, 10-20 or 15-20 nucleotides. The second spacer may be a polyadenine having a length of about 1-2, 1-3, 1-4, 1-5, 1-6, 1-7, 1-8, 1-9, 1-10, 1-15, 1-20, 2-3, 2-4, 2-5, 2-6, 2-7, 2-8, 2-9, 2-10, 2-15, 2-20, 3-4, 3-5, 3-6, 3-7, 3-8, 3-9, 3-10, 3-15, 1-20, 4-5, 4-6, 4-7, 4-8, 4-9, 4-10, 4-15, 4-20, 5-6, 5-7, 5-8, 5-9, 5-10, 5-15, 5-20, 6-7, 6-8, 6-9, 6-10, 6-20, 7-8, 7-9, 7-10, 7-15, 7-20, 8-9, 8-10, 8-15, 8-20, 9-10, 9-15, 9-20, 10-15, 10-20 or 15-20 nucleotides. The first spacer and the second spacer may be the same. The first spacer and the second spacer may be different. In one embodiment, the first spacer is a polyadenine having 5-7 nucleotides and the second spacer is a polyadenine having 4-6 nucleotides.

The kit may provide a liquid comprising the first primer and the second primer at a combined concentration of about 0.1-100, 0.1-500, 0.1-750, 0.1-1,000, 0.1-5,000, 0.1-10,000, 1-100, 1-500, 1-750, 1-1,000, 1-5,000, 1-10,000, 5-100, 5-500, 5-750, 5-1,000, 5-5,000, 5-10,000, 10-100, 10-500, 10-750, 10-1,000, 10-5,000, 10-10,000, 15-100, 15-500, 15-750, 15-1,000, 15-5,000, 15-10,000, 20-100, 20-500, 20-750, 20-1,000, 20-5,000, 20-10,000, 100-500, 100-750, 100-1,000, 100-5,000, 100-10,000, 250-500, 250-750, 250-1,000, 250-5,000, 250-10,000, 500-750, 500-1,000, 500-5,000, 500-10,000, 1,00-5,000 or 1,000-10,000 nM.

In the kit, the substrate may be selected from the group consisting of luminol, 2,2′-azino-bis(3-ethylbenzothiazoline-6-sulfonic acid) (ABTS) and 3,3′,5,5′-tetramethylbenzidine (TMB). For example, the substrate may be 2,2′-azino-bis(3-ethylbenzothiazoline-6-sulfonic acid) (ABTS).

The present invention provides a composition for detecting a target DNA sequence. The composition comprises a first primer and a second primer. The first primer comprises a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer. The second primer comprises a second protector, the HRPzyme sequence, a second spacer and a reverse primer sequence for the target DNA sequence in the order from the 5′ end to the 3′ end of the second primer.

According to the composition of the present invention, the target DNA sequence may be a gene or a fragment thereof specific to a pathogen. The pathogen may be a bacterium, for example, Vibrio parahaemolyticus, E. coli, S. aureus, or S. enterica. The gene may be the tlh gene specific to Vibrio parahaemolyticus, the 16s rRNA gene specific to E. coli, the nuc A gene specific to S. aureus, or the inv A gene specific to S. enterica. In one embodiment, the target DNA sequence is the tlh gene or a fragment thereof specific to Vibrio parahaemolyticus, and presence of the tlh gene or a fragment thereof in a sample indicates the presence of the Vibrio parahaemolyticus in the sample.

In the composition, the forward primer sequence may consist of a DNA sequence at the 5′ end of the target DNA sequence while the reverse primer sequence may consist of a DNA sequence complementary to a DNA sequence at the 3′ end of the target DNA sequence. The forward primer sequence may have 12-30 nucleotides. The reverse primer sequence may have 12-30 nucleotides. Where the target DNA sequence is the tlh gene or a fragment thereof, the forward primer sequence may be ACTCAACACAAGAAGAGATCGACAA (SEQ ID NO: 2) and the reverse primer sequence may be GATGAGCGGTTGATGTCCAAA (SEQ ID NO: 3) (FIG. 8).

In the composition, the first protector may be a polyadenine having about 1-5, 1-10, 1-15, 1-20, 1-25, 1-30, 1-35, 1-40, 1-45, 1-50, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45 or 20-50 nucleotides. The second protector may be a polyadenine having about 1-5, 1-10, 1-15, 1-20, 1-25, 1-30, 1-35, 1-40, 1-45, 1-50, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45 or 20-50 nucleotides. The first protector and the second protector may be the same. The first protector and the second protector may be different.

In the composition, the first spacer may be a polyadenine having a length of about 1-2, 1-3, 1-4, 1-5, 1-6, 1-7, 1-8, 1-9, 1-10, 1-15, 1-20, 2-3, 2-4, 2-5, 2-6, 2-7, 2-8, 2-9, 2-10, 2-15, 2-20, 3-4, 3-5, 3-6, 3-7, 3-8, 3-9, 3-10, 3-15, 1-20, 4-5, 4-6, 4-7, 4-8, 4-9, 4-10, 4-15, 4-20, 5-6, 5-7, 5-8, 5-9, 5-10, 5-15, 5-20, 6-7, 6-8, 6-9, 6-10, 6-20, 7-8, 7-9, 7-10, 7-15, 7-20, 8-9, 8-10, 8-15, 8-20, 9-10, 9-15, 9-20, 10-15, 10-20 or 15-20 nucleotides. The second spacer may be a polyadenine having a length of about 1-2, 1-3, 1-4, 1-5, 1-6, 1-7, 1-8, 1-9, 1-10, 1-15, 1-20, 2-3, 2-4, 2-5, 2-6, 2-7, 2-8, 2-9, 2-10, 2-15, 2-20, 3-4, 3-5, 3-6, 3-7, 3-8, 3-9, 3-10, 3-15, 1-20, 4-5, 4-6, 4-7, 4-8, 4-9, 4-10, 4-15, 4-20, 5-6, 5-7, 5-8, 5-9, 5-10, 5-15, 5-20, 6-7, 6-8, 6-9, 6-10, 6-20, 7-8, 7-9, 7-10, 7-15, 7-20, 8-9, 8-10, 8-15, 8-20, 9-10, 9-15, 9-20, 10-15, 10-20 or 15-20 nucleotides. The first spacer and the second spacer may be the same. The first spacer and the second spacer may be different. In one embodiment, the first spacer is a polyadenine having 5-7 nucleotides and the second spacer is a polyadenine having 4-6 nucleotides.

In the composition, the first primer and the second primer may have a combined concentration of about 0.1-100, 0.1-500, 0.1-750, 0.1-1,000, 0.1-5,000, 0.1-10,000, 1-100, 1-500, 1-750, 1-1,000, 1-5,000, 1-10,000, 5-100, 5-500, 5-750, 5-1,000, 5-5,000, 5-10,000, 10-100, 10-500, 10-750, 10-1,000, 10-5,000, 10-10,000, 15-100, 15-500, 15-750, 15-1,000, 15-5,000, 15-10,000, 20-100, 20-500, 20-750, 20-1,000, 20-5,000, 20-10,000, 100-500, 100-750, 100-1,000, 100-5,000, 100-10,000, 250-500, 250-750, 250-1,000, 250-5,000, 250-10,000, 500-750, 500-1,000, 500-5,000, 500-10,000, 1,00-5,000 or 1,000-10,000 nM.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Utilizing HRPzyme, a Cost-Effective Vibrio parahaemolyticus Detection Method

Vibrio parahaemolyticus is a prominent infectious bacterium responsible for causing widespread cases of acute gastroenteritis in humans globally. In this regard, colorimetric detection can be essentially used as a sensitive, rapid, and cost-effective detection method. In our research, we have developed a PCR-based detection platform integrated with HRPzyme and utilizing DNAzyme as a signaling probe which mimics peroxidase activity. The colorimetric signal is detectable at concentrations as low as 101 cfu mL-1 when measured with a spectrophotometer and at 10³ cfu mL-1 through visual inspection. Additionally, extending the polyadenine length to 10 nucleotides resulted in a significant reduction in the background signaling of HRPzyme activity, yielding a relative intensity of 3.07±0.23 arbitrary units (a.u.). Notably, even after a 120-minute incubation period, there were no further changes observed in the colorimetric signal in positive samples, maintaining a consistent relative intensity of OD₄₁₀=0.55±0.08.

### 1. MATERIALS AND METHODS

### 1.1 Reagents and Equipment

We obtained Q5 high-fidelity DNA polymerase (M0491) and a 100-bp ladder (B7025) from New England BioLabs (NEB). All primers used in this study were purchased from Integrated DNA Technologies (Coralville, IA, USA). DNA amplification was conducted using a Bio-Rad thermocycler (T100), and gel electrophoresis was carried out on a 2% w/v agarose gel (Thermo Scientific, 17852) prepared with 1× trisboric acid/EDTA (TAE) buffer. We employed GelRed Nucleic Acid Gel Stain from Biotium, Inc., USA, for DNA staining. In addition to conventional PCR, we employed Real-time PCR (RT-PCR) using Power SYBR Green PCR Master Mix (Applied Biosystems, 4367659) for the molecular detection and confirmation of V. parahaemolyticus. ABTS, Hemin, and hydrogen peroxide (H₂O₂) were purchased from the Sigma-Aldrich (USA), and the Colorimetric assay test was performed using a microplate spectrophotometer device (Bio-Rad, Hercules, CA, USA).

### 1.2 Microorganisms Culture and Positive Samples Selection

V. parahaemolyticus (ATCC 49529, tlh+) was cultured on Thiosulfate-citrate-bile salts-sucrose (TCBS) selective agar (Thermo Scientific, R454752) at 37° C. The resulting colonies were then molecularly confirmed using specific primers for 16S rRNA and tlh regions. We collected samples using a sterile plastic inoculating loop and resuspended them in 1× phosphate-buffered saline (PBS) to create bacterial suspensions containing 10° to 107 colony-forming units per milliliter (cfu mL-1). We also conducted a specificity test by comparing V. parahaemolyticus with five other bacterial species. The treatments were carried out in alignment with our prior unpublished study and the findings documented by Lee and colleagues in 2020. These bacteria, namely Klebsiella pneumoniae (ATCC 13883), Escherichia coli (ATCC 43889), Pseudomonas aeruginosa (ATCC 10145), and Staphylococcus aureus (ATCC 14154), were cultured on LB media (Thermo Scientific, 22700025) at 37° C. However, Shewanella algae (ATCC 51192) was grown on Marin agar (2216E) at 30° C., and Vibrio cholerae (ATCC 14035) was grown on (TCBS) media at 37° C. We selected these bacterial species due to their frequent presence in seafood products.

### 1.3 Primer Design and Molecular Confirmation of the Vibrio parahaemolyticus

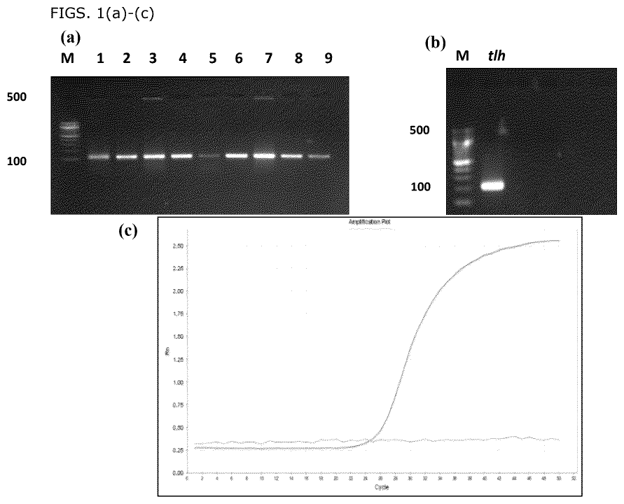

We used two sets of primers for the molecular confirmation of V. parahaemolyticus: 16S rRNA (F: GGCGTAAAGCGCATGCAGGT (SEQ ID NO: 4), R: GAAATTCTACCCCCCTCTACAG (SEQ ID NO: 5) and tlh (F: ACTCAACACAAGAAGAGATCGACAA (SEQ ID NO: 2); R: GATGAGCGGTTGATGTCCAAA (SEQ ID NO: 3). The PCR were performed under the following conditions: (1) 16S rRNA: denaturation at 95° C. for 3 min, followed by 34 cycles of denaturation at 95° C. for 30 sec, annealing at 58° C. for 30 sec, and primer extension at 72° C. for 1 min; followed by final extension at 72° C. for 5 min. (2) tlh: denaturation at 95° C. for 3 min, followed by 35 cycles of denaturation at 95° C. for 30 sec, annealing at 60° C. for 40 sec, and primer extension at 72° C. for 1 min; followed by final extension at 72° C. for min (FIG. 1).

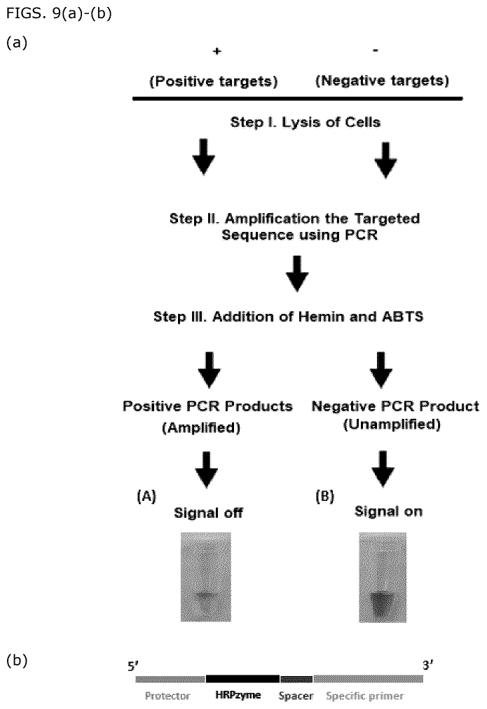

We obtained the sequence data of the tlh gene in V. parahaemolyticus from the NCBI GenBank (FIG. 8). Using SnapGene software (version 6.0), we designed the 10 forward and reverse primers, which included a protector, HRPzyme sequence, spacer, and the complementary sequence to the targeted region in the tlh gene (FIGS. 9a-b). In order to prevent unwanted folding of the HRPzyme sequence (GGGTAGGGCGGGTTGGGT) (SEQ ID NO: 1) into a double strand in the presence of Hemin and to minimize background signals, we introduced various lengths of protector sequences (poly A) at the 5′ end of both the forward and reverse tlh primers. These lengths ranged from 0 to 20 nucleotides, as detailed in Table 1.

TABLE 1
Primer's information used for
Colorimetric PCR-Based detection of
Vibrio parahaemolyticus in this study.

| Primers | |
|---|---|
| Name | Primers sequences (5′-3′) |
| HRP-Vib-F0 | GGGTAGGGCGGGTTGGGTAAAAAACTCAACACAA |
| | GAAGAGATCGACAA |
| HRP-Vib-F0 | GGGTAGGGCGGGTTGGGTAAAAAGATGAGCGGTT |
| | GATGTCCAAA |
| HRP-Vib-F5 | AAAAAGGGTAGGGCGGGTTGGGTAAAAAACTCAA |
| | CACAAGAAGAGATCGACAA |
| HRP-Vib-F5 | AAAAAGGGTAGGGCGGGTTGGGTAAAAAGATGAG |
| | CGGTTGATGTCCAAA |
| HRP-Vib-F10 | AAAAAAAAAAGGGTAGGGCGGGTTGGGTAAAAAA |
| | CTCAACACAAGAAGAGATCGACAA |
| HRP-Vib-F10 | AAAAAAAAAAGGGTAGGGCGGGTTGGGTAAAAAG |
| | ATGAGCGGTTGATGTCCAAA |
| HRP-Vib-F15 | AAAAAAAAAAAAAAAGGGTAGGGCGGGTTGGGTA |
| | AAAAACTCAACACAAGAAGAGATCGACAA |
| HRP-Vib-F15 | AAAAAAAAAAAAAAAGGGTAGGGCGGGTTGGGTA |
| | AAAAGATGAGCGGTTGATGTCCAAA |
| HRP-Vib-F20 | AAAAAAAAAAAAAAAAAAAAGGGTAGGGCGGGTT |
| | GGGTAAAAAACTCAACACAAGAAGAGATCGACAA |
| HRP-Vib-F20 | AAAAAAAAAAAAAAAAAAAAGGGTAGGGCGGGTT |
| | GGGTAAAAAGATGAGCGGTTGATGTCCAAA |

Note:
Protector sequences: Italic-green colored sequences (on the 5′ end of the bolded sequence);
HRPzyme sequence: Bold-black colored sequence;
spacer: underlined-red colored sequences,
specific primer: Blue colored (on the 3′ end of the underlined sequence).
HRP-Vib-F0: GGGTAGGGCGGGTTGGGTAAAAAACTCAACACAAGAAGAGATCGACAA (SEQ ID NO: 8);
HRP-Vib-R0: GGGTAGGGCGGGTTGGGTAAAAAGATGAGCGGTTGATGTCCAAA (SEQ ID NO: 9);
HRP-Vib-F5: AAAAAGGGTAGGGCGGGTTGGGTAAAAAACTCAACACAAGAAGAGATCGACAA (SEQ ID NO: 10);
HRP-Vib-R5: AAAAAGGGTAGGGCGGGTTGGGTAAAAAGATGAGCGGTTGATGTCCAAA (SEQ ID NO: 11);
HRP-Vib-F10: AAAAAAAAAAGGGTAGGGCGGGTTGGGTAAAAAACTCAACACAAGAAGAGATCGACAA (SEQ ID NO: 12);
HRP-Vib-R10: AAAAAAAAAAGGGTAGGGCGGGTTGGGTAAAAAGATGAGCGGTTGATGTCCAAA (SEQ ID NO: 13);
HRP-Vib-F15: AAAAAAAAAAAAAAAGGGTAGGGCGGGTTGGGTAAAAAACTCAACACAAGAAGAGATCGACAA (SEQ ID NO: 14);
HRP-Vib-R0: AAAAAAAAAAAAAAGGGTAGGGCGGGTTGGGTAAAAAGATGAGCGGTTGATGTCCAAA (SEQ ID NO: 15);
HRP-Vib-F20: AAAAAAAAAAAAAAAGGGTAGGGCGGGTTGGGTAAAAAACTCAACACAAGAAGAGATCGACAA (SEQ ID NO: 16);
HRP-Vib-R20: AAAAAAAAAAAAAAAGGGTAGGGCGGGTTGGGTAAAAAGATGAGCGGTTGATGTCCAAA (SEQ ID NO: 17).

In the next stage, we confirmed the amplification of the targeted region using specific HRPzyme-integrated primers through electrophoresis on a 2% agarose gel. To initiate the reaction, we mixed 8 μL of the PCR product with 5 L of 500 UM hemin dissolved in MES buffer (containing 1% DMSO, 0.05% Triton X-100, 25 mM MES, 200 mM NaCl, 10 mM KCl, pH=5.1) and allowed it to incubate for 30 minutes at room temperature. Subsequently, we added 50 μL of 10 mM ABTS, 2 μL of H₂O₂ (30%), and 1 mL of Citrate buffer (pH=4). The reaction's absorbance was measured at OD=410 nm.

To optimize the HRPzyme-colorimetric reaction, we explored different primer concentrations (ranging from 15 to 750 nM), annealing temperatures (ranging from 40 to 65° C.), and reaction times (ranging from 5 to 60 minutes). These investigations were conducted using a concentration of 104 cfu mL-1 of V. parahaemolyticus, and data presented based on 6 independent experiments (replicates=3).

### 1.4 Sensitivity and the Specificity of the Assay for Detection of V. parahaemolyticus

We assessed the sensitivity of the colorimetric assay (OD=410 nm) using a concentration range of V. parahaemolyticus spanning from 10° to 107 cfu mL-1. Subsequently, we compared these results with those obtained from the gel electrophoresis-based assay. We also conducted specificity testing by evaluating the assay's performance against different pathogenic microorganisms, including K. pneumoniae, E. coli, P. aeruginosa, S. aureus, S. algae, Vibrio cholerae, and V. parahaemolyticus. To confirm the results of the colorimetric assay, we also utilized gel electrophoresis (2%).

### 1.5. Detection of Vibrio parahaemolyticus in Crassostrea Virginica and Callinectes sapidus Samples

We collected C. virginica specimens (n=10) from one of our monitoring sites located at Latitude 38° 38.613′ N and Longitude 075° 07.631′ W in Rehoboth Beach, Delaware, USA. After collection, the C. virginica specimens were thoroughly washed with deionized water. Subsequently, various samples were extracted from the following areas: (1) adductor muscle, (2) heart, (3) pericardial cavity, (4) gonad area, and (5) gill parts. In the subsequent step, we took 100 mg from each of the samples, mixed them, and then subjected the mixture to homogenization and vortexing for 1 minute with 1 mL of phosphate buffer (PBS, 1 mM, pH=7.4), and 100 μL of the supernatant (centrifuged at 5000 rpm, 2 min) was used for subsequent analysis.

We also collected C. sapidus specimens (n=10) from one of our study sites situated at Latitude: 38° 38.613′ N and Longitude: 75° 07.55′ W in Rehoboth Beach, Delaware, USA. To extract the hemolymph of C. sapidus, we used a 1 mL 27 Gauge syringe with a needle. Subsequently, 100 mg of the hemolymph was mixed with 100 μL PBS buffer (1 mM, pH=7.4), vortexed for 1 minute, and the resulting supernatant (obtained after centrifugation at 5000 rpm for 2 minutes) was utilized for subsequent analysis. We employed a protocol outlined by Rizvi and Bej (Antonie Van Leeuwenhoek, 98, 279-290 (2010)) to confirm the presence of V. parahaemolyticus. This confirmation was achieved using RT-PCR instruments targeting the tlh region. The sizes of the amplicons were subsequently verified by running them on a 2% (w/v) agarose gel alongside a 100-bp ladder. Data presented based on 10 independent experiments (replicates=3).

### 2. RESULTS AND DISCUSSION

### 2.1 Designing an HRPzyme-Integrated PCR-Based Platform for the Colorimetric Detection of V. Parahaemolyticus

Following the lysis of V. parahaemolyticus, we conducted one-step PCR using the designed HRPzyme-integrated primers. Subsequently, we confirmed the PCR amplification through gel electrophoresis. We also examined the design of our HRPzyme-based primers for the formation of primer dimers in non-template samples by visualizing the PCR product on a 2% agarose gel.

According to our design, the HRPzyme sequence present in the primer was blocked during PCR by forming double-stranded DNA. At the end of the PCR, the double-stranded HRPzyme sequence was prevented from folding into a G-quadruplex structure in the presence of hemin. This prevented the HRPzyme from catalyzing the oxidation of ABTS, which is necessary to produce a green-blue color in the presence of hemin and H₂O₂.

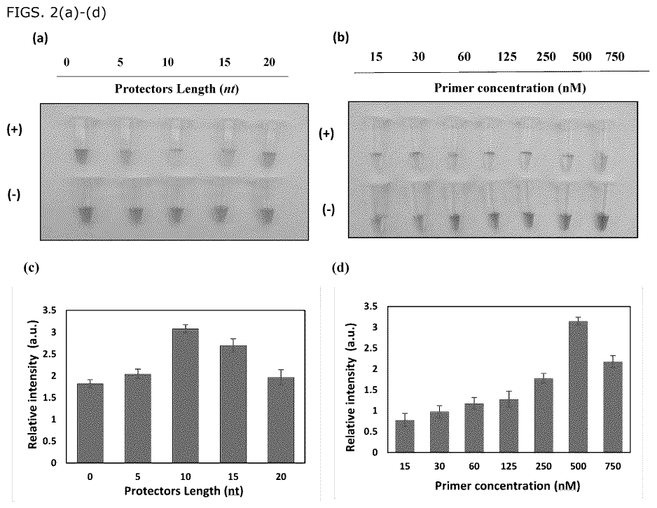

Additionally, unamplified primers folded in the presence of hemin, leading to the oxidation of ABTS and the production of a green-blue colored product when H₂O₂ was added (FIG. 2). As a result, the colorimetric signal inversely correlated with the presence of gene-specific PCR products, being at its maximum in the absence of the target. This enabled us to detect PCR products generated from V. parahaemolyticus DNA either through visual observation or by using a UV-visible spectrophotometer.”

After incubating the PCR product from the tlh gene with hemin and ABTS, followed by the addition of H₂O₂ (30%), we observed a colorimetric signal ranging from dark to light green-blue in the amplified products. Notably, there were no noticeable differences in the colorimetric signal among protector lengths of 0, 5, and 20 nucleotides after an 80-minute incubation period. This observation highlights the significant role of protector length in preventing background signals.

Expanding the polyadenine length to 10 nucleotides led to a substantial reduction in the background signaling of HRPzyme activity, resulting in a relative intensity of 3.07±0.23 arbitrary units (a.u.). This change allowed us to distinguish a light green-blue color compared to other protector lengths. However, increasing the polyadenine length to 20 nucleotides had the opposite effect, resulting in a reduced relative intensity of 1.96±0.17 a.u. Thus, we selected HRPzyme-integrated primers containing a 10 nt polyadenine sequence as the protector for the subsequent steps of the study (FIG. 2d).

In our experiment, we employed protector and spacer sequences in conjunction with HRPzyme and the complementary sequences designed for the specific targeting of gene sequences. The primary purpose of the protector and spacer sequences was to block the folding of the HRPzyme sequence when it formed a double-stranded configuration, thus it could effectively prevent false-positive reactions. The HRPzyme sequence exhibited peroxidase-like activity by forming a G-quadruplex complex in the presence of hemin. This G-quadruplex complex, originating from the HRPzyme, catalyzed the oxidation of the ABTS substrate in the presence of hydrogen peroxide, resulting in the development of color. In HRPzyme-based detection assays, the length of the protector has consistently been a major concern for researchers seeking to manage background signals generated by HRPzyme sequence activity.

### 2.2 Optimization the Colorimetric Detection of the Amplicons

The colorimetric conditions for detecting PCR products were optimized through different experiments involving both negative and positive samples. Colorimetric signals were assessed using both a spectrophotometer and visual observation with the naked eye.

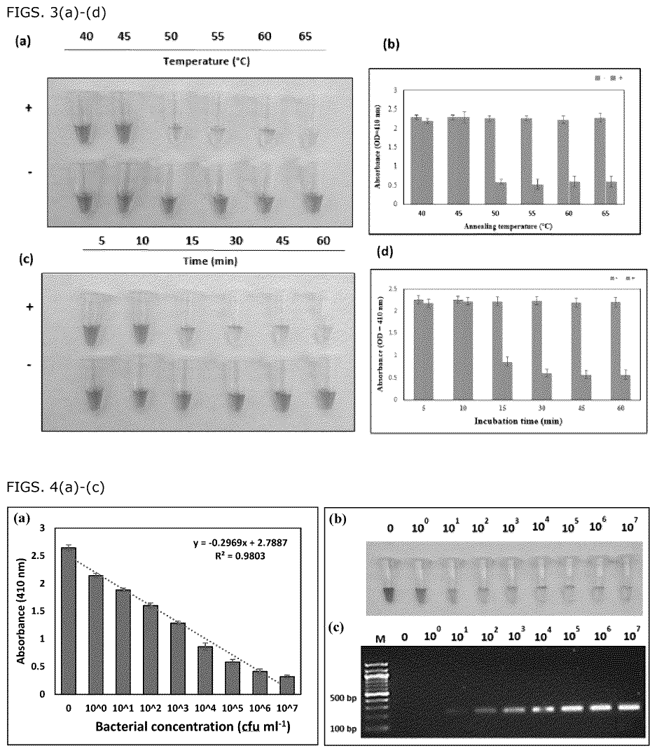

In the initial step, we investigated the impact of annealing temperature (Ta) on the detection of colorimetric signals in our HRPzyme-integrated PCR assay. As shown in FIG. 3. no detectable difference in colorimetric signals were observed between incubation of the positive samples at 40 and 45° C. and the negative samples. However, the signal showed a significant improvement compared to the negative control when we increased the amplification temperature to 65° C. This indicates that the designed primers were less efficient in binding to the targeted region at lower temperatures (<50° C.), whereas the higher temperature, up to 65° C., facilitated primer binding, resulting in more reproducible amplification of the targeted region on the tlh gene. We did not detect any significant differences in the colorimetric signals between 6° and 65° C. Nevertheless, for the subsequent stages of the study, we selected an amplification temperature of 60° C. to prevent the possibility of secondary annealing (FIG. 3). Annealing temperature is a critical step in amplifying the targeted genomic region and addressing the issue of non-specific amplification. During the annealing stage, it is vital to maintain a temperature that allows for the binding of primers to the target DNA. However, it's equally important that, the temperature is not set too low (≤45° C.), as this can lead to the formation of undesired, non-specific duplexes or intramolecular hairpin structures, both of which can result in false-positive results. Meantime, the higher the temperature is the primer require longer compatible sequence to bind to and as a result your specificity will be higher, since the higher annealing temperatures (>65° C.) possess greater thermal energy and have the potential to interfere with hydrogen bonds, which can hinder primer-template binding. Finding the right balance in annealing temperature is essential for accurate and specific amplification. Various G-quadruplex-based assays have recently been developed for pathogen detection using a range of annealing temperatures, typically from 55 to 65° C.

We also found that, the incubation time significantly influences the development of the colorimetric signal. There was no significant difference in the development of the colorimetric signal between 5- and 10-minute incubation times for both positive and negative samples (reaction time: 45 minutes). However, the signal was enhanced when the incubation time was extended to 60 minutes (OD₄₁₀=0.57±0.1). Furthermore, no further alteration in the colorimetric signal in positive samples was observed even after 120 minutes of incubation (relative intensity=OD₄₁₀=0.55±0.08).

The amplification time is a critical parameter that needs to be optimized in G-quadruplex-based assays to ensure accurate and reliable results. The goal of optimization is to strike a balance between achieving sufficient amplification of the target G-quadruplex structure and minimizing the risk of non-specific amplification or primer dimers. We also examined the impact of different concentrations of HRPzyme-integrated primers (ranging from 15 to 750 nM, with a 10 nt protector length), and observed a concentration-dependent influence on the colorimetric signal when comparing positive and negative samples.

At primer concentrations ranging from 15-30 nM, we did not observe a significant signal (indicated by a light green-blue color), which could potentially lead to false colorimetric signals. However, when we increased the primer concentration to 500 nM, we observed a substantial impact on the colorimetric signal in both positive and negative samples.

To ensure the prevention of false-positive results, we utilized a primer concentration of 500 nM, which yielded a relative intensity of 3.08±0.09 a.u. for HRPzyme-integrated primers in the subsequent phases of our study. Prior research has bolded the essential role of primer concentration in modulating the colorimetric signals produced by G-quadruplex DNAzymes. Distinguishing between the colorimetric signals of negative and positive samples becomes challenging at lower concentrations of HRPzyme-integrated primers. Conversely, higher primer concentrations may introduce an excess of free primers, potentially leading to false-positive color development.

2.3 The sensitivity and specificity of the PCR-based platform integrated with HRPzyme for colorimetric detection of V. parahaemolyticus.

We employed the following optimized amplification conditions for assessing the sensitivity and specificity of the introduced HRPzyme-integrated PCR platform for the detection of V. parahaemolyticus: denaturation at 95° C. for 3 minutes, an incubation period at 60° C. for 30 minutes, followed by 30 cycles of denaturation at 95° C. for 15 seconds, annealing at 60° C. for 10 seconds, and primer extension at 72° C. for 10 seconds, concluding with a final extension step at 72° C. for 5 minutes. To evaluate the sensitivity of the assay, a series of V. parahaemolyticus dilutions (ranging from 0 to 107 cfu mL-1) were prepared in 1×PBS buffer.

The amplification of the targeted tlh gene region was carried out using a PCR thermocycler, and the success of the PCR amplification was confirmed through agarose gel electrophoresis to eliminate the possibility of false-positive signals. As illustrated in FIG. 4, the band intensities observed in the gel electrophoresis directly correlated with the concentration of V. parahaemolyticus. Additionally, the colorimetric signals displayed an inverse relationship with the concentration of the tested V. parahaemolyticus, allowing for clear differentiation from the negative control.

The linear relationship between various concentrations of V. parahaemolyticus and the optical density (OD=410 nm) showed a strong correlation (R²=0.98). Our findings indicate that, the colorimetric signals were inversely proportional to the concentration of the tested V. parahaemolyticus, allowing for clear differentiation from the negative control. Notably, even at lower concentrations, the colorimetric signal was detectable using a spectrophotometer, becoming visible at a concentration as low as 10³ cfu mL-1 (OD₄₁₀: 1.3±0.2).

In the specificity test, a variety of pathogens, including K. pneumoniae, E. Coli, P. aeruginosa, S. aureus, S. algae, and V. cholerae were examined alongside V. parahaemolyticus using the optimized HRPzyme-integrated PCR assay. These pathogens were chosen due to their common occurrence in seafood products. To provide an additional layer of confirmation, the PCR products were subjected to gel electrophoresis, allowing for a comparison between the colorimetric signals obtained and the positive bands generated.

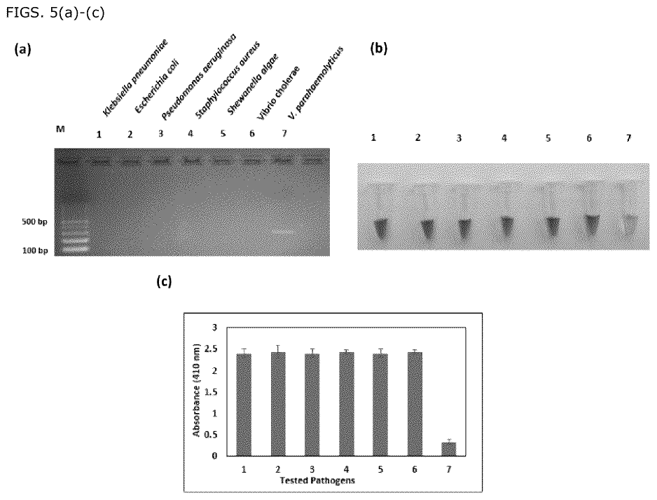

As illustrated in FIG. 5, among the array of pathogens examined, a positive colorimetric signal was exclusively detected for V. parahaemolyticus (OD₄₁₀: 0.4±0.03). This outcome was subsequently validated through both PCR and RT-PCR, as shown in FIG. 5. These results unequivocally establish the remarkable specificity of the HRPzyme-integrated PCR assay, specifically designed for the precise detection of the target pathogen.

G-quadruplex-based assays and PCR techniques are frequently utilized for pathogen detection, with a substantial body of evidence indicating that G-quadruplex-based assays offer high sensitivity and specificity. A recent Meta-Analysis on the available reports shows the PCR and G-quadruplex based assays has the sensitivity 95.6% and 96.6% and specificity of 98.7% and 97.6%, respectively.

Our results also demonstrate that, the proposed assay exhibits a sensitivity comparable to the PCR approach. The colorimetric signal can be detected at a concentration as low as 101 cfu mL-1 using a spectrophotometer and at 10³ cfu mL-1 by the naked eye. However, when the test results fall within a range lower than 10³ CFU mL-1, the positive identification outcomes using the HRPzyme-integrated PCR-based platform are diminished. Therefore, we recommend the use of culture-based methods for V. parahaemolyticus identification as a complementary approach. 2.4 Validation of the HRPzyme-integrated PCR-based colorimetric platform for the detection of V. parahaemolyticus in C. virginica and C. sapidus. To evaluate the applicability and reliability of our test, we applied our proposed HRPzyme-integrated PCR-based platform to detect V. parahaemolyticus in Eastern oysters (C. virginica) and blue crabs (C. sapidus). In this context, various samples were gathered from the muscle, heart, pericardial cavity, gonad area, and gills of the C. virginica. Positive colorimetric signals were then compared to the outcomes obtained through gel electrophoresis and the RT-PCR method.

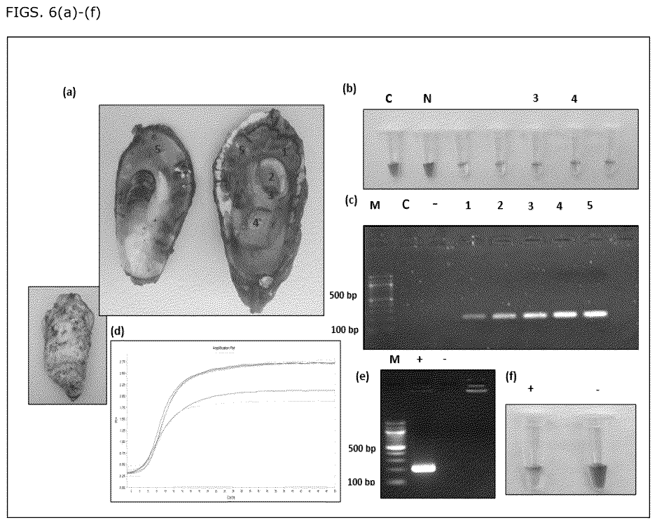

As illustrated in FIG. 6, a distinct colorimetric signal (OD₄₁₀=0.43±0.02) was evident in the organs tested, indicating contamination with V. parahaemolyticus in the C. virginica samples. These findings were further validated through PCR and RT-PCR. Notably, no amplified bands were observed in the negative agarose gel electrophoresis of the V. parahaemolyticus samples, whereas a specific band of the correct size was consistently obtained in the gel electrophoresis results.

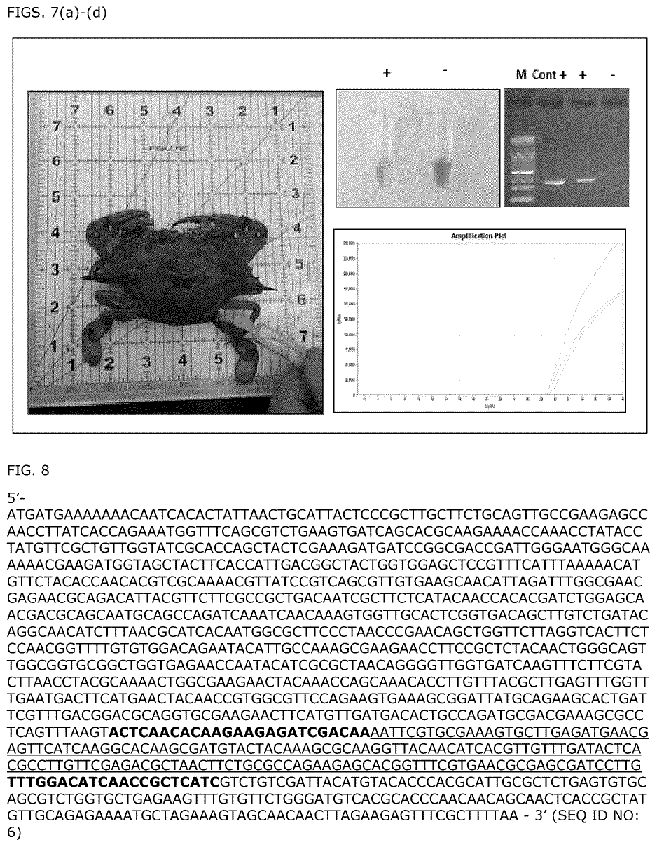

To assess the test's validity in C. sapidus, hemolymph from both V. parahaemolyticus-positive and -negative samples was extracted and compared. As depicted in FIG. 7, the colorimetric signal generated by our proposed HRPzyme-integrated PCR-based platform was distinctly observed in the V. parahaemolyticus-positive samples (OD₄₁₀=0.5±0.03). These results were consistently supported by gel electrophoresis and RT-PCR analysis, providing parallel confirmation. These findings strongly endorse the specificity of the HRPzyme-integrated PCR assay for detecting the targeted pathogen, enabling the identification of V. parahaemolyticus in seafood products. The assay's specificity relies on the precise primers integrated with HRPzyme for the targeted tlh sequence, enabling the detection of this specific foodborne pathogen.

Owing to diverse impacts of the food products on the public health, the safety of the food products must be the main concern during the harvesting phase. In this regard, there is an essential need for accurate, sensitive, fast, and straightforward molecular biology-based techniques to detect the pathogens however the specificity of the primers will play the major role, for example, RdRP and BPO genes were employed for detection of different genogroup of noroviruses, ORF-1ab and N-gene were used for detection of SARS-COV-2 and here by this study we suggest targeting specific region on tlh gene for detection of V. parahaemolyticus using the HRPzyme-integrated PCR assay. However, it's essential to keep in consideration that, the quantity of targeted DNA can change significantly among different samples. This variety presents a notable challenge when attempting to establish uniform diagnostic performance criteria, particularly with specific sample types. Additionally, distinguishing between active infection and asymptomatic colonization poses a challenge when using the HRPzyme-integrated PCR-based platform because the method does not differentiate between live and dead cells. Hence, it is essential to take these limitations into account when using this method as a diagnostic tool and to employ a complementary approach as needed.

### 3. CONCLUSION

The global consumption of seafood products has witnessed a significant increase, emphasizing the critical importance of promptly detecting pathogens like V. parahaemolyticus. Thus, accurate, rapid and straight forward methods for high-through put screening of pathogens is a necessary demand. In this study, we have introduced a HRPzyme-integrated PCR based platform for colorimetric detection of V. parahaemolyticus targeting a specific region on the tlh gene to form G-quadruplex DNAzymes. In this study, we utilized the G-quadruplex loop region to create a binding site for hemin, resulting in the formation of a catalytic G-quadruplex/hemin DNAzyme. The catalytic cycle of this DNAzyme is initiated by the peroxidase enzyme, and the colorimetric HRPzyme-integrated PCR detection platform was optimized with 500 nM forward and reversed primers and Ta of 60° C. and reaction time of 60 min.

The method may detect the V. parahaemolyticus at the concentration of 10 cfu mL⁻³ by naked-eye and can be accomplished within 2 hours. In contrast to the existing PCR and RT-PCR techniques our approach showed several advantages such as rapid V. parahaemolyticus detection, increase cost effectiveness, and being eco-friendly by eliminating the usage of toxic fluorescence dyes, such as ethidium bromide (Table 2).

TABLE 2
Comparison of the developed HRPzyme-Based Vibrio
parahaemolyticus. detection with Other Methods.

| | Detection | Detection | | |
|---|---|---|---|---|
| | time | range | | |
| Method | (h) | (cfu ml⁻¹) | Characteristics | References |
| 1 Streak Plate/probe method | <72 | 10⁵-10⁰ | Lengthy | Blackstone et |
| | | | and costly | al. (2003) |
| 2 Real time PCR method | <4 | 10⁵-10⁰ | Lengthy | Costa, |
| | | | and costly | Ferreira, |
| | | | | Simões, Silva, |
| | | | | and Campos |
| | | | | (2022) |
| 3 PCR based | <2 | 10⁵-10⁰ | Costly | Bonny et al. |
| | | | | (2022) |
| 4 Gold nanoparticle-based | <3 | 10³-10⁰ | Lengthy | Wu et al. |
| enzyme-linked antibody | | | and costly | (2014) |
| 5 Magnetic nanoparticles | <12 | 10¹ | Lengthy | Blank-Shim et |
| | | | and costly | al. (2017) |
| 6 CE-RAA-CRISPR Assay | <5 | 6.7 × 10¹ | Lengthy | Lv et al. |
| | | | and costly | (2022) |
| 7 Visual Detection of V. | 30 min | 10² | Costly | Jiang et al. |
| parahaemolyticus using Combined | | | | (2022) |
| CRISPR/Cas12a and Recombinase | | | | |
| Polymerase Amplification | | | | |
| 8 Duplex droplet digital PCR | 30 min | 8.15 × 10¹ | Costly | Zhou et al. |
| combined with propidium | | | | (2023) |
| monoazide | | | | |
| 9 HRPzyme based targeting tlh | 2 | 10³ | Rapid and | This study |
| region | | | cheap | |

### REFERENCES

Blackstone, G. M., Nordstrom, J. L., Vickery, M. C., Bowen, M. D., Meyer, R. F., & DePaola, A. (2003). Detection of pathogenic Vibrio parahaemolyticus in oyster enrichments by real time PCR. Journal of Microbiological Methods, 53, 149-155. https://doi.org/10.1016/S0167-7012 (03) 00020-4.
Costa, C., Ferreira, G. D., Simões, M., Silva, J. L., & Campos, M. J. (2022). Real-time PCR protocol for detection and quantification of three pathogenic members of the Vibrionaceae family. Microorganisms, 10, Article 2060. https://doi.org/10.3390/microorganisms10102060.
Bonny, S. Q., Hossain, M. M., Uddin, S. M. K., Pulingam, T., Sagadevan, S., & Johan, M. R. (2022). Current trends in polymerase chain reaction-based detection of three major human pathogenic vibrios. Critical Reviews in Food Science and Nutrition, 62, 1317-1335. https://doi.org/10.1080/10408398.2020.1841728.
Blank-Shim, S. A., Schwaminger, S. P., Borkowska-Panek, M., Anand, P., Yamin, P., Fraga-García, P., et al. (2017). Binding patterns of homo-peptides on bare magnetic nanoparticles: Insights into environmental dependence. Scientific Reports, 7, Article 14047. https://doi.org/10.1038/s41598-017-13928-6.
Lv, X., Cao, W., Zhang, H., Zhang, Y., Shi, L., & Ye, L. (2022). CE-RAA-CRISPR assay: A rapid and sensitive method for detecting Vibrio parahaemolyticus in seafood. Foods, 11, Article 1681. https://doi.org/10.3390/foods11121681.
Jiang, H. J., Rong, T. A. N., Min, J. I. N., Jing, Y. I. N., Gao, Z. X., Li, H. B., et al. (2022). Visual detection of Vibrio parahaemolyticus using combined CRISPR/Cas12a and recombinase polymerase amplification. Biomedical and Environmental Sciences, 35, 518-527. https://doi.org/10.3967/bes2022.069.
Zhou, H., Liu, X., Lu, Z., Hu, A., Ma, W., Shi, C., et al. (2023). Quantitative detection of Vibrio parahaemolyticus in aquatic products by duplex droplet digital PCR combined with propidium monoazide. Food Control, 144, Article 109353. https://doi.org/10.1016/j.foodcont.2022.109353.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for detecting a target DNA polynucleotide in a sample, comprising:
(a) obtaining a target DNA polynucleotide from a sample and a control DNA polynucleotide, wherein the target DNA polynucleotide comprises a target DNA sequence, and wherein the control DNA polynucleotide lacks the target DNA sequence;
(b) providing a first primer and a second primer, wherein the first primer comprises a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer, wherein the second primer comprises a second protector, the HRPzyme sequence, a second spacer and a reverse primer sequence for the target DNA sequence in the order from the 5′ end to the 3′ end of the second primer;
(c) performing a polymerase chain reaction (PCR) in a test mixture, wherein the test mixture comprises the target DNA polynucleotide, the first primer, and the second primer, whereby a PRC product is produced in the test mixture;
(d) exposing the test mixture from step (c) to hemin, a substrate and hydrogen peroxide;
(e) making a test color measurement in the test mixture from step (d);
(f) performing a PCR in a control mixture, wherein the control mixture comprises the control DNA polynucleotide, the first primer, and the second primer, whereby no PRC product is produced in the control mixture;
(g) exposing the control mixture from step (f) to hemin, a substrate and hydrogen peroxide;
(h) making a control color measurement in the control mixture from step (g); and
(i) comparing the test color measurement with the control color measurement, wherein a color difference between the test color measurement and the control color measurement indicates the presence of the target DNA polynucleotide in the sample.

2. The method of claim 1, wherein the target DNA sequence is a gene or a fragment thereof specific to a bacterium.

3. The method of claim 2, wherein the gene is the tlh gene specific to Vibrio parahaemolyticus.

4. The method of claim 3, wherein the forward primer sequence is ACTCAACACAAGAAGAGATCGACAA (SEQ ID NO: 2) and the reverse primer sequence is GATGAGCGGTTGATGTCCAAA (SEQ ID NO: 3).

5. The method of claim 1, wherein each of the first protector and the second protector is independently a polyadenine having 1-50 nucleotides.

6. The method of claim 1, wherein the first protector and the second protector are the same.

7. The method of claim 1, wherein each of the first spacer and the second spacer is independently a polyadenine having a length of 1-20 nucleotides.

8. The method of claim 1, wherein the first spacer is a polyadenine having 5-7 nucleotides and the second spacer is a polyadenine having 4-6 nucleotides.

9. The method of claim 1, wherein the test mixture comprises the first primer and the second primer at a combined concentration of 1-1000 nM before the PCR reaction is performed in the test mixture.

10. The method of claim 1, wherein the substrate is selected from the group consisting of luminol, 2,2′-azino-bis(3-ethylbenzothiazoline-6-sulfonic acid) (ABTS) and 3,3′,5,5′-tetramethylbenzidine (TMB).

11. The method of claim 10, wherein the substrate is 2,2′-azino-bis(3-ethylbenzothiazoline-6-sulfonic acid) (ABTS).

12. The method of claim 1, wherein the test sample comprises Vibrio parahaemolyticus at a concentration of 1-100 colony-forming units (CFU) per mL.

13. The method of claim 1, wherein the sample is obtained from an organism.

14. The method of claim 13, wherein the organism is Crassostrea virginica.

15. The method of claim 13, wherein the organism is Callinectes sapidus.

16. The method of claim 1, further comprising detecting the color change by a naked eye.

17. The method of claim 1, further comprising detecting the color change within 0.5-3 hours after the target DNA polynucleotide is obtained from the sample.

18. A kit for detecting a target DNA polynucleotide in a sample, wherein the target DNA polynucleotide comprises a target DNA sequence, comprising:
(a) a first primer comprising a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer,
(b) a second primer comprising a second protector, the HRPzyme sequence, a second spacer and a reverse primer sequence for the target DNA sequence in the order from the 5′ end to the 3′ end of the second primer, and
(c) an instruction for performing the method of claim 1.

19. The kit of claim 18, further comprising one or more agents selected from the group consisting of a control DNA polynucleotide, hemin, a substrate, and hydrogen peroxide.

20. A composition for detecting a target DNA sequence, comprising a first primer and a second primer, wherein the first primer comprises a first protector, the HRPzyme sequence of GGGTAGGGCGGGTTGGGT (SEQ ID NO: 1), a first spacer and a forward primer sequence for the target DNA sequence in the order from the 5′ end to the 3′ end of the first primer, wherein the second primer comprises a second protector, the HRPzyme sequence, a second spacer and a reverse primer sequence for the target DNA sequence in the order from the 5′ end to the 3′ end of the second primer.

21-26. (canceled)
